(19) United States
(12) Patent Application Publication
(10) Pub. No.: US 2006/0206203 A1
(43) Pub. Date: Sep. 14, 2006

(54) Valvular support prosthesis

(76) Inventors: Jun Yang, Dove Canyon, CA (US); Frank M. Zeng, Irvine, CA (US)

Correspondence Address: Raymond Sun, 12420 Woodhall Way, Tustin, CA, 92782, US

(21) Appl. No.: 11/373,401

(22) Filed: Mar. 10, 2006

Related U.S. Application Data
(60) Provisional application No. 60/660,649, filed on Mar. 10, 2005.

(51) Int. Cl. A61F 2/24

(52) U.S. Cl. 623/2.37

(57) ABSTRACT

A ring prosthesis provides suitable flexibilities/stiffness three-dimensionally at various points about the circumference of an associated heart valve, and is shaped proportionally to fit about the annulus of the associated heart valve. The ring prosthesis also provides a certain flexibility to conform to the natural non-planar shape of the annulus (e.g., saddle shape for mitral valve surface) with or without preformation of the ring prosthesis. The prosthesis can also be used as an artificial annulus for further valve anchoring.

### BACKGROUND OF THE INVENTION

1. Field of the Invention

The present invention relates to a support prosthesis for use in medical applications, and in particular, to an annuloplasty ring that is adapted for use in supporting a heart valve.

2. Description of the Prior Art

Annuloplasty rings for use as heart valve prostheses are well known in adult patients. Most such annuloplasty rings are substantially planar. Recently, an interest in non-planar (e.g., saddle-shaped) annuloplasty rings has developed. The conventional non-planar annuloplasty rings tend to be substantially rigid throughout the annuloplasty ring. Unfortunately, uniformly rigid annuloplasty rings do not conform to the natural non-planar shape of the human valve annulus. As a result, these uniformly rigid annuloplasty rings do not move with the valve tissue, thereby increasing the stress to the leaflet or surrounding tissue.

In addition, many patients who suffer from disfunction of the mitral and/or tricuspid valves(s) of the heart, surgical repair of the valve (i.e., “valvuloplasty”) is a desirable alternative to valve replacement. One problem associated with the annuloplasty rings of the prior art is that when such annuloplasty rings are implanted into children or adolescents (such as pediatric patients with CVA or RVD), the subsequent growth of the patient may render the annuloplasty ring too small for its intended function, thereby abnormally constricting the annulus. Follow-up surgery would be necessary to replace the originally implanted annuloplasty ring with a larger annuloplasty ring suitable for the then-current size of the patient. However, the tissue of the heart valve annulus grows into the fabric of the annuloplasty ring by design, so that the annuloplasty ring is soon embedded in living tissue, thereby making such replacement surgery problematic.

### SUMMARY OF THE DISCLOSURE

It is an object of the present invention to provide a ring prosthesis that has varying flexibility to conform to the natural non-planar shape of a human valve annulus.

It is another object of the present invention to provide a ring prosthesis that reduces stress to the leaflet and surrounding tissue due to annuloplasty.

It is yet another object of the present invention to provide an expandable annuloplasty ring for implantation in a heart valve annulus.

In order to accomplish the objects of the present invention, the present invention provides an annuloplasty ring having a frame member that has varying three-dimensional flexibility/expandability at different regions of the frame member. The ring also includes a suture-permeable outer layer that covers the frame member, and a soft sleeve surrounding the frame member.

The ring prosthesis according to the present invention is also adapted to expand upon natural growth of the patient's annulus, or upon application of a dilatation force surgically applied. The outer layer can be provided in the form of a fabric covering that is preferably radially expandable. The ring prosthesis may also be implanted percutaneously and secured to the dilated natural human valve annulus.

According to the present invention, the ring prosthesis provides suitable flexibilities/stiffness three-dimensionally at various points about the circumference of an associated heart valve, and is shaped proportionally to fit about the annulus of the associated heart valve. The ring prosthesis also provides a certain flexibility to conform to the natural non-planar shape of the annulus (e.g., saddle shape for mitral valve surface) with or without preformation of the ring prosthesis. The prosthesis can also be used as an artificial annulus for further valve anchoring.

### BRIEF DESCRIPTION OF THE DRAWINGS

FIG. 1 is a top plan view of one embodiment of a mitral annuloplasty ring prosthesis according to the present invention, with the covering material and insert being partially removed to expose the individual segments.

FIGS. 2(a), 2(b) and 2(c) are enlarged cross-sectional views of the section A-A′ of the ring prosthesis of FIG. 1 according to different embodiments thereof.

FIG. 3 is a perspective view of one embodiment of the frame member for the ring prosthesis of FIG. 1.

FIG. 4 illustrates a conventional saddle-shape mitral annulus.

FIG. 5 is a bottom view of another embodiment of a mitral annuloplasty ring prosthesis according to FIG. 2(b) of the present invention, which has a wire or drawstring running through the sleeve.

FIG. 6 is a side view of the ring prosthesis of FIG. 1 pre-formed to the natural mitral valve shape (e.g., saddle shape).

FIG. 7 is a perspective view of yet another embodiment of a mitral annuloplasty ring prosthesis according to the present invention, with the covering material and insert being partially removed to expose the individual segments.

FIG. 8 is a perspective view of the frame member of the ring prosthesis of FIG. 7.

FIG. 9 is the perspective view of the ring prosthesis of FIGS. 7-8 implanted on the top of a natural mitral valve.

FIG. 10 is a perspective view of the frame member of FIG. 3 modified to include interlocking elements.

FIGS. 11(a)-11(c) illustrate how different types of interlocking struts can be used in connection with the frame member of FIG. 10.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

Inasmuch as the human mitral valve is far more likely to require repair than the tricuspid, aortic and pulmonary valves, the description of the present invention herein will be based on the repair of a mitral valve. However, the same principles discussed herein in connection with the repair of a mitral valve also apply to the repair of all other heart valves.

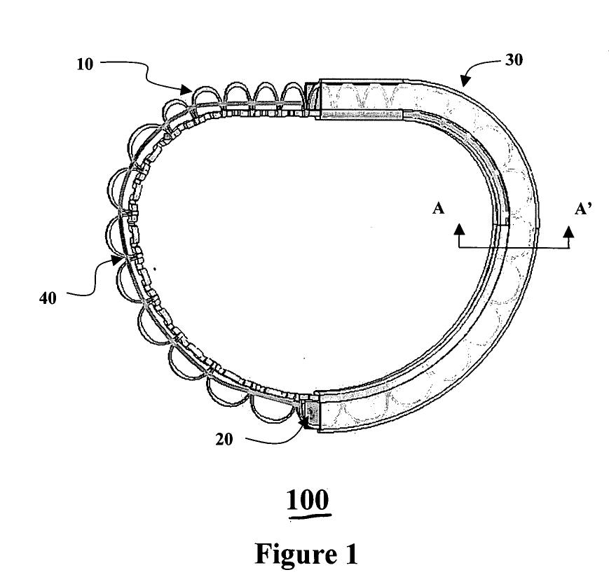

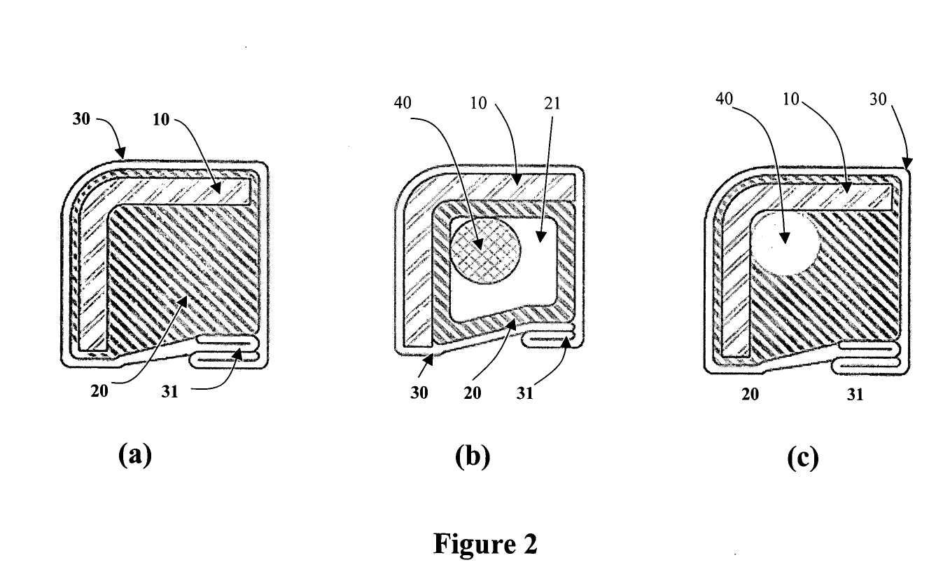

FIGS. 1-3 illustrate a ring prosthesis 100 according to one embodiment of the present invention. The ring prosthesis 100 is illustrated herein as an annuloplasty ring 100. The annuloplasty ring 100 can be made of a single inner frame member 10 that is covered by a suture-permeable outer layer 30, and which has a soft expandable insert or sleeve 20 (hereinafter “insert”) surrounding the frame member 10. This is best shown in FIG. 2(a), which shows the frame member 10 completely embedded in a soft insert 20, and then further covered by a biocompatible material as the outer layer 30. The covering material for the outer layer 30 may be folded and sealed, as shown at 31. The ring prosthesis 100 can be secured to a valve annulus by suture or staples.

The frame member 10 can be made of a material having shape memory, such as Nitinol. The frame member 10 can also be made of other biocompatible materials, such as Cobalt-Chromium alloys, and titanium alloys. The structural pattern for the frame member 10 can be cut from a flat sheet or tube and then heat or cold formed into a three-dimensional shape.

The biocompatible material for the outer layer 30 can be made from a suture-permeable material such as tissue, Dacron or ePTFE cloth, or other synthetic material that allows selected expansion of the insert 20 and frame member 10. The soft expandable insert 20 can be made of silicone, cotton, and other similar biocompatible filling materials.

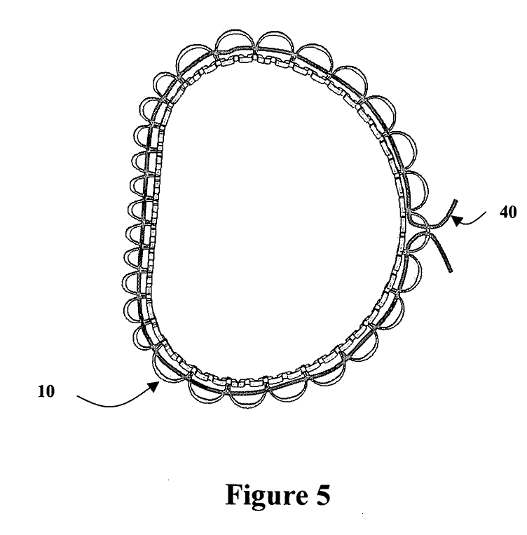

As shown in FIGS. 2(b) and 5, the ring prosthesis 100 can optionally include a wire 40 that extends adjacent to, and along the circumference of, the frame member 10. The wire 40 can be a metallic wire or a drawstring, and may be used to control or restrict the dimension of the metallic frame member 10 either permanently or temporarily. Specifically, if the wire 40 is biodegradable, it is disappear after a period of time after implantation, so that the restriction of the dimension is temporary. Conversely, if the wire 40 is not biodegradable, then the restriction of the dimension is permanent. FIG. 2(b) shows the wire 40 tied to the outside of the ring prosthesis 100 to restrict the expansion of the ring prosthesis 100. In the embodiment of FIG. 2(b), the soft insert 20 can be positioned so that it only partially covers the frame member 10, and defines an opened inner space 21 that receives the wire 40.

If a drawstring 40 is used, the drawstring 40 extends through the inner space 21 of the insert 20 and can be pulled or released to constrict and remodel the orifice of the ring prosthesis 100 so as to secure the ring prosthesis 100 in place at a valve annulus. The two ends of the drawstring 40 may be tied during the surgical procedure. The drawstring 40 can be made of non-stretchable wire or tape, and also can be made of an elastic material, such as silicone. The constriction applied to the frame may be permanent or temporary, as described above.

FIG. 2(c) illustrates an alternative embodiment where both the frame member 10 and the restriction wire 40 are completely embedded in the soft insert 20.

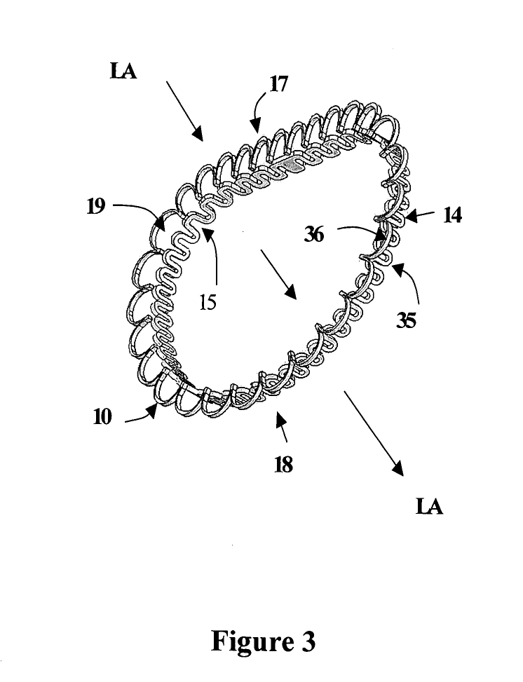

FIG. 3 illustrates the frame member 10 of the mitral annuloplasty ring 100 of FIG. 1. The shape of the frame member 10 is designed based on the natural planar shape of a valve annulus, which is a slight D shape for a mitral valve. The frame member 10 may be flat or pre-formed into a saddle shape. The expandability and flexibility may not be uniform throughout the frame member 10. Specifically, the expandability and flexibility of the frame member 10 at each location can be designed to match the need for that particular location. For example, regions 14, 17 and 18 can have different expandabilities and flexibilities because the natural human mitral valve has different anatomies and movement dynamics at the anterior, posterior and side regions. This variable expandability and flexibility is achieved by providing different structural patterns, or varying the thickness of the material within the prosthesis (e.g., the frame member 10 and the insert 20), which allows certain movement within its elasticity.

The present invention provides different ways for varying the flexibility and expandability of the prosthesis ring 100. In this regard, it is the construction of the frame member 10 which allows the ring prosthesis 100 to experience bending and deformation in three dimensions. In a first example, as shown in FIG. 3, the frame member 10 defines a thin-walled tubular member configured with a pattern of alternating struts or zig-zags 35 that define a plurality of slots 15 formed therebetween, with the slots 15 being disposed substantially parallel to the longitudinal axis LA of the tubular ring 100. The slots 15 can be formed by cutting away portions of the material that is used for the frame member 10. It is the provision of a pattern of structural modifications to the frame member 10 (such as, but not limited to, the zig-zags 35, slots 15 and/or the cells 19 described below) which allows the annuloplasty ring 100 to be bent during cardiac cycles, and to conform to the non-planar surface of the valve annulus.

As another example, the frame member 10 defines a thin-walled tubular member that has a plurality of cells 19 formed therein. The cells 19 may be deformed to allow the annuloplasty ring 100 to expand upon stretching by circumferential external forces. The cells 19 can be formed by cutting material away from the frame member 10 to form openings that make up the cells 19.

On the top of the frame member 10 in FIG. 3, additional material may be bent outwardly to form a flange 36 to facilitate the securing of the ring prosthesis 100 to the valve annulus. The flange 36 can be made up of a plurality of U-shaped elements. The flange 36 can be used to further control the flexibility of the annuloplasty ring 100. For example, configuring the flange 36 with a greater number of U-shaped elements will cause the frame menber 10 to be less flexible, while configuring the flange 36 with a lesser number of U-shaped elements will cause the frame menber 10 to be more flexible.

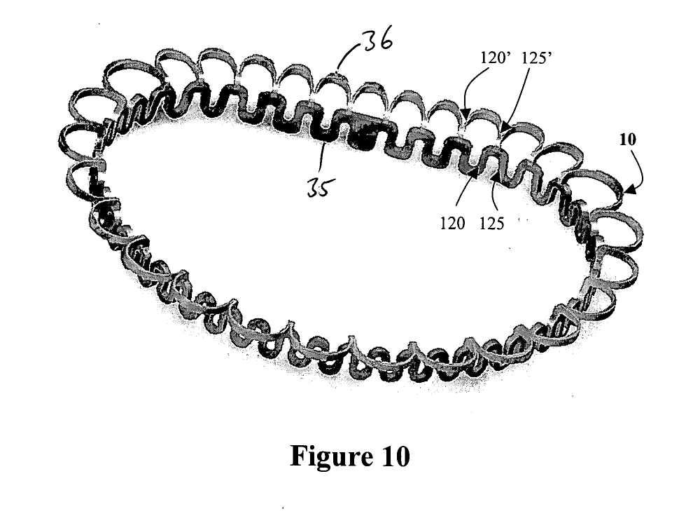

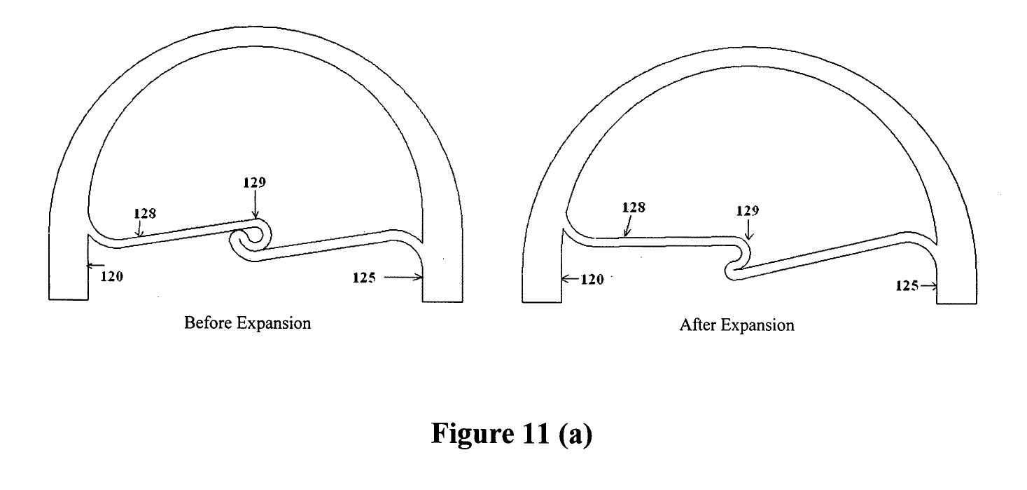

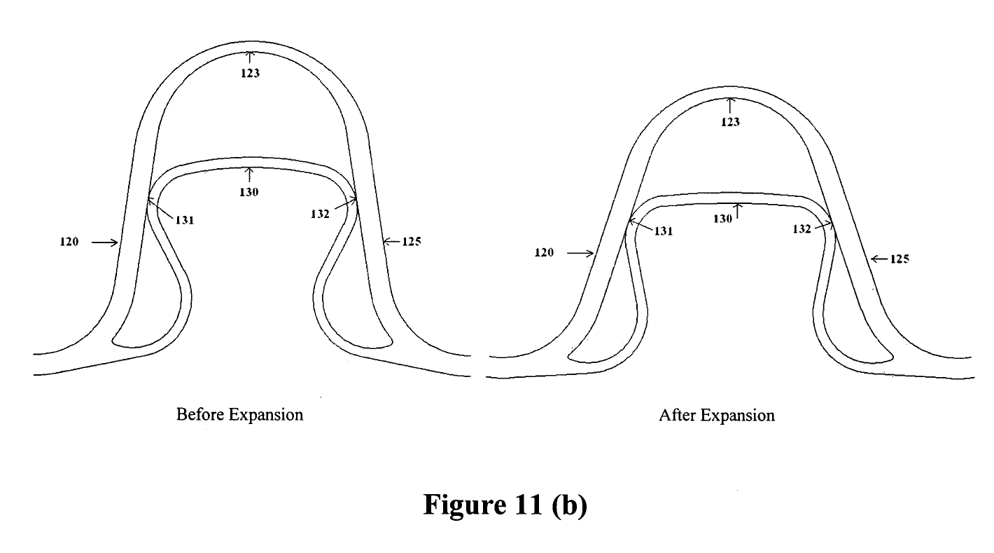

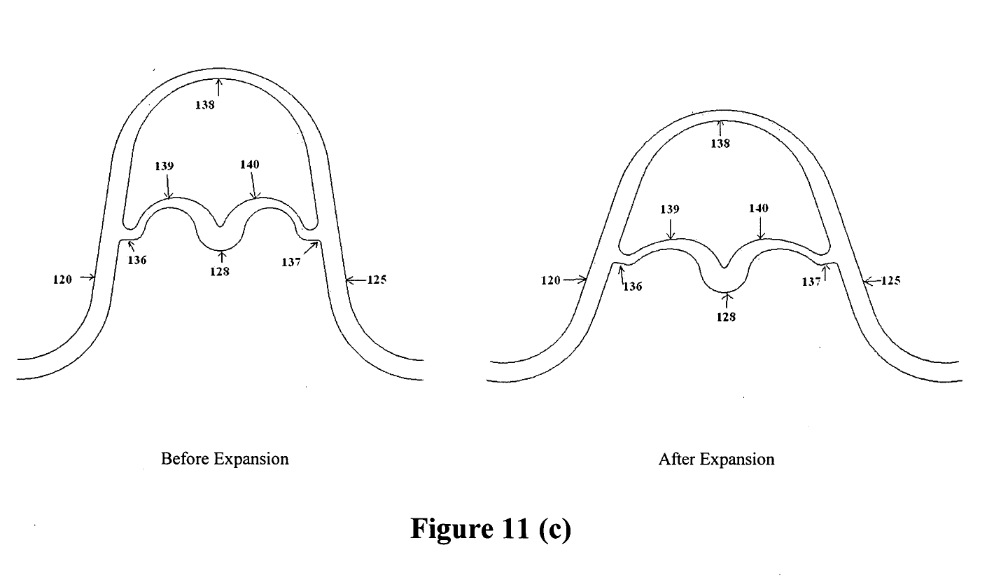

In addition to the cells 19 and slots 15 described above, additional structures can be provided to vary the flexibility and expandability of the ring prosthesis 100, to cause structural deformation, or to function as locking mechanisms to prevent the retraction of the structure of the frame member 10 when there are no external stretching forces (e.g., when the natural valve annulus is not expanding). Additional interlocking struts or bars shaped as arcs, zig-zags, and similar alternating elements may be added to the top or bottom of the frame member 10. For example, FIGS. 10 and 11(a)-10(c) illustrate how additional struts can be provided on the frame member 10 to function as one-way locking mechanisms that prevent the retraction of the frame member 10.

Referring first to FIG. 10, locations 120 and 125 can be provided on the zig-zags 35 of the frame member 10, and locations 120′ and 125′ can be provided on the flange 36, for receiving the struts described in connection with FIGS. 11(a)-11(c). These struts can have the same material as the frame member 10, and can even be cut from the material used to form the frame member 10. These struts can have different configurations or patterns to obtain the desired flexibility, expandability and anti-retraction for the ring prosthesis 100. For example, in FIG. 11(a), the strut 129 has a center coil that is unwrapped permanently during the expansion (i.e., outward motion) of the locations 120 and 125 of the zig-zag 35. This expansion of the locations 120, 125 allows the segment 128 to rotate into a more horizontal position, thereby making it more difficult for the locations 120, 125 to return to their original positions. In FIG. 11(b), the expansion of the locations 120, 125 pulls on the strut 130, causing the strut 130 to straighten itself to form a straight segment. The resisting forces applied by the strut 130 against the zig-zag 35 at the locations 131 and 132 will make it more difficult for the locations 120, 125 to return to their original positions. In FIG. 11(c), the expansion of the locations 120, 125 pulls on the strut segments 136, 137, 139 and 140, causing them to form a larger radius segment. The resisting forces applied by the strut segments 136 and 137 against the locations 120, 125 will make it more difficult for the struts 120, 125 to return to their original positions. These struts can also function to vary the flexibility and expandability of the ring prosthesis 100 because their provision or absence at certain locations of the ring prosthesis 100 will cause these locations to be more rigid (where the struts are provided) or more flexible (where the struts are absent).

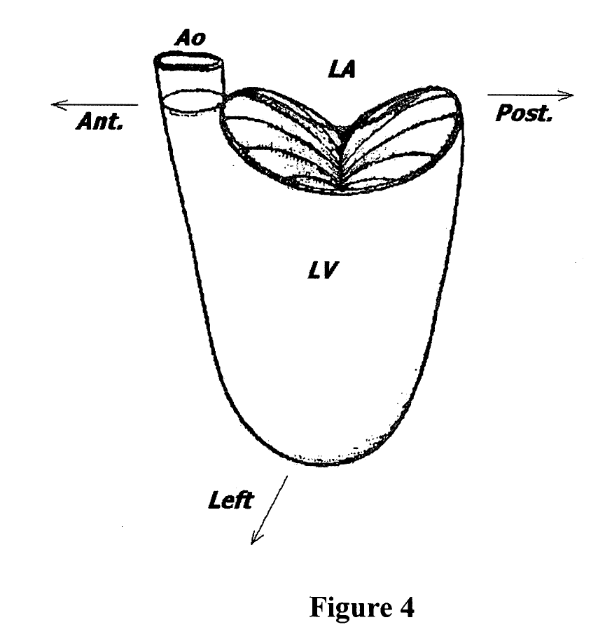

FIG. 4 shows the mitral annular shape of a natural valve. Mitral leaflets concave towards the left ventricle during systolic pressure. The leaflets form a saddle shape.

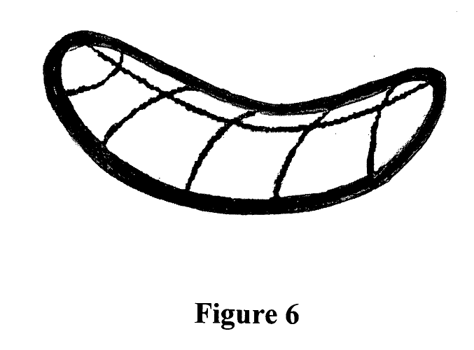

FIG. 6 is a side view of the mitral annuloplasty ring prosthesis 100 pre-formed to the natural mitral valve shape (saddle shape).

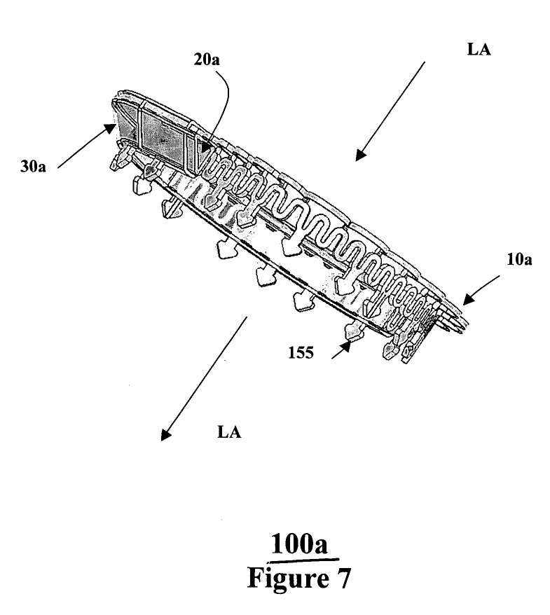

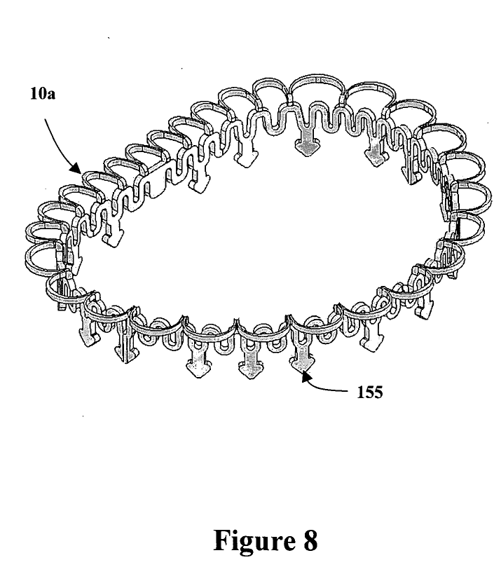

FIG. 7 illustrates a modification that can be made to the mitral annuloplasty ring prosthesis 100, where the ring prosthesis 100a can be the same as the ring prosthesis 100 except that hooks 155 are now provided to anchor the ring prosthesis 100a to the annulus of the natural valve. The frame member 10a of the ring prosthesis 100a is shown in greater detail in FIG. 8, with the hooks 155 extending in the longitudinal direction LA.

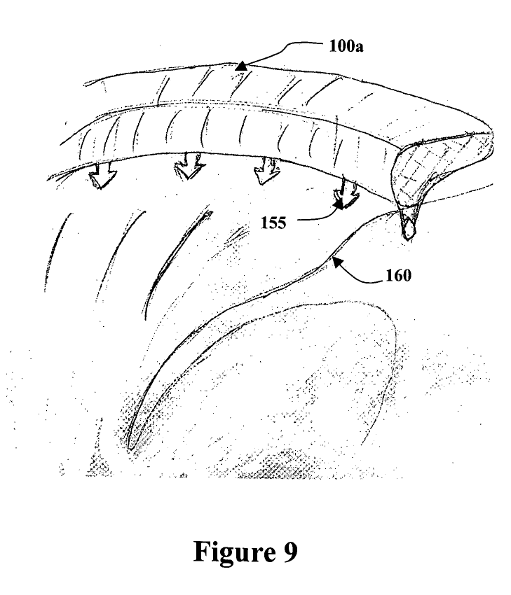

The ring prosthesis 100/100a can be implanted percutaneously. To carry out the percutaneous procedure, the ring prosthesis 100/100a is delivered to the valve annulus by a catheter or other known delivery means, and then mechanically expanded to the size of the dilated natural valve annulus by means of a holder and/or a balloon using techniques that are well-known in the art. The expansion of the ring prosthesis 100/100a is tailored to the expanded natural valve annulus; for example, greater expansion in the posterior section of the mitral valve annulus than in the anterior section because the anterior section does not change much in a diseased case or during the growth of a pediatric patient. If hooks 155 are provided, the ring prosthesis 100a is then attached on top of the annulus 160 by means of the hooks 155 that are specifically placed to allow the ring prosthesis 100a to adapt to the three-dimensional shape of the annulus. See FIG. 9. Otherwise, the ring prosthesis 100 can be stapled or sutured to the valve annulus 160. As the mechanical expansion force is removed, the ring prosthesis 100/100a returns to its unexpanded dimension, which in turn reshapes the dilated natural annulus. The retraction forces of the ring prosthesis 100/100a around its circumference are designed to have different values to provide the optimum reshaping of the annulus.

The ring prosthesis 100/100a may be used as an artificial annulus for anchoring or receiving future artificial valve (e.g., a self-expanding heart valve) that is to be deployed within the ring prosthesis 100/100a.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. An annuloplasty ring, comprising:
a frame member that has varying three-dimensional flexibility/expandability at different regions of the frame member;
a suture-permeable outer layer that covers the frame member; and
a soft insert surrounding the frame member.

2. The apparatus of claim 1, wherein the frame member is completely embedded in the insert.

3. The apparatus of claim 1, wherein the frame member is positioned adjacent the insert.

4. The apparatus of claim 1, wherein the outer layer 30 is be folded and sealed.

5. The apparatus of claim 1, wherein the frame member is made of a material having shape memory.

6. The apparatus of claim 1, wherein the frame member is cut from a flat sheet of material and then heat formed into a three-dimensional shape.

7. The apparatus of claim 1, further including a wire that extends adjacent to, and along the circumference of, the frame member.

8. The apparatus of claim 7, wherein the insert partially covers the frame member and defines an opened inner space that receives the wire.

9. The apparatus of claim 7, wherein the wire is biodegradable.

10. The apparatus of claim 7, wherein the wire is biostable.

11. The apparatus of claim 1, wherein the frame member includes a plurality of slots.

12. The apparatus of claim 1, wherein the frame member includes a plurality of cells.

13. The apparatus of claim 11, wherein the frame member includes a plurality of cells.

14. The apparatus of claim 1, wherein the frame member has a pattern that includes a plurality of alternating elements.

15. The apparatus of claim 1, wherein the frame member includes a plurality of hooks extending longitudinally from the frame member.

16. The apparatus of claim 1, wherein the frame member is formed of a pattern that provides varying flexibilities/expandability three-dimensionally at different locations about the circumference of the frame member.

17. The apparatus of claim 1, wherein the frame member includes a plurality of interlocking struts.
